(19) United States
(12) Patent Application Publication
(10) Pub. No.: US 2012/0095302 A1
(43) Pub. Date: Apr. 19, 2012

(54) USING TV AS HEALTH MONITOR

(75) Inventor: Suranjit Adhikari, San Diego, CA (US)

(73) Assignee: SONY CORPORATION

(21) Appl. No.: 12/904,419

(22) Filed: Oct. 14, 2010

(51) Int. Cl. A61B 5/00

(52) U.S. Cl. 600/301

(57) ABSTRACT

A TV can receive user health information from a health monitoring sensor and enable a user to view a user interface on the TV responsive to current and/or historical user health information received by the health monitoring sensor. The TV may also communicate through the Internet with a user's health care provider to send user health information to the health care provider.

### I. FIELD OF THE INVENTION

The present application relates generally to using televisions (TVs) as health monitors.

### II. BACKGROUND OF THE INVENTION

Internet access through TVs is typically provided by essentially programming the TV as though it were a computer executing a browser. As understood herein, such devices can be leveraged for many novel uses owing to their connectivity to the Internet.

As also understood herein, with an aging populace it is important to provide people, particularly the elderly, with an easy, intuitive means to track their health. In this way, they can better adjust their habits, meals, etc. to achieve a healthy lifestyle. Present principles seek to leverage TV technology, which is readily understood and frequently used by people, to assist in enabling people to monitor their health and provide health information to health care providers in an efficient and easy manner.

### SUMMARY OF THE INVENTION

Accordingly, a TV includes a housing, a display on the housing, a TV tuner, and a processor in the housing controlling the display and TV tuner. An input device can communicate with the processor and may have a health monitoring sensor attached thereto. The processor may execute logic including presenting a first user interface (UI) on the display which is responsive to user health information gathered by the health monitoring sensor and received from the input device.

The health monitoring sensor may be a band or bracelet configured to be worn on a person's wrist in non-limiting embodiments. The input device, which may be a remote control in non-limiting embodiments, can have an interface which converts user health information gathered by the health monitoring sensor to infrared (IR) signals accepted by the TV. Further, the user health information may include various measurements relating to a user's health, including, but not limited to, heart rate data, blood pressure data, blood glucose data and body mass index (BMI) data.

The processor can cause a first UI to present a visual representation of user health information such as heart rate data, blood pressure data, blood glucose data and/or BMI data. Also, user health information may be stored in a storage area of the TV.

Further, the processor in the TV can cause a second UI to be presented on the display, wherein the second UI may present a visual representation of user health information history associated with stored user health information such as, but not limited to, heart rate data, blood pressure data, blood glucose data and BMI data.

Additionally, in certain non-limiting embodiments, the TV may be an internet protocol TV which can have a network interface so that the processor may communicate with the Internet through the network interface. The internet protocol TV can either store user health information for future use by a health care provider or send current and/or stored user health information to a user's health care provider through the Internet.

In another aspect, a method includes receiving user health information from a health monitoring sensor at a TV. The method also includes storing the user health information. A visualization of the user's health information history is presented on the TV.

In another aspect, an apparatus includes a video display, a TV tuner and a processor controlling the display and TV tuner and communicating with the Internet through the network interface. An input device communicates with the processor and has a health monitoring sensor attached it. The processor executes logic, including

receiving user health information from the health monitoring sensor and processing the user health information. The logic also includes presenting a visualization of the user health information on the video display.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a block diagram of a non-limiting example system in accordance with present principles;

FIG. 2 is a flow chart of non-limiting logic for presenting user health information on a first UI.

FIG. 3 is a flow chart of non-limiting logic for presenting user health information history on a second UI.

FIG. 4 is a flow chart of non-limiting logic for sending user health information to a health care provider.

FIGS. 5 and 6 are non-limiting example screen shots of UIs that can be presented on the TV in accordance with present principles for enabling a person to monitor his or her health.

### DETAILED DESCRIPTION OF THE PREFERRED EMBODIMENT

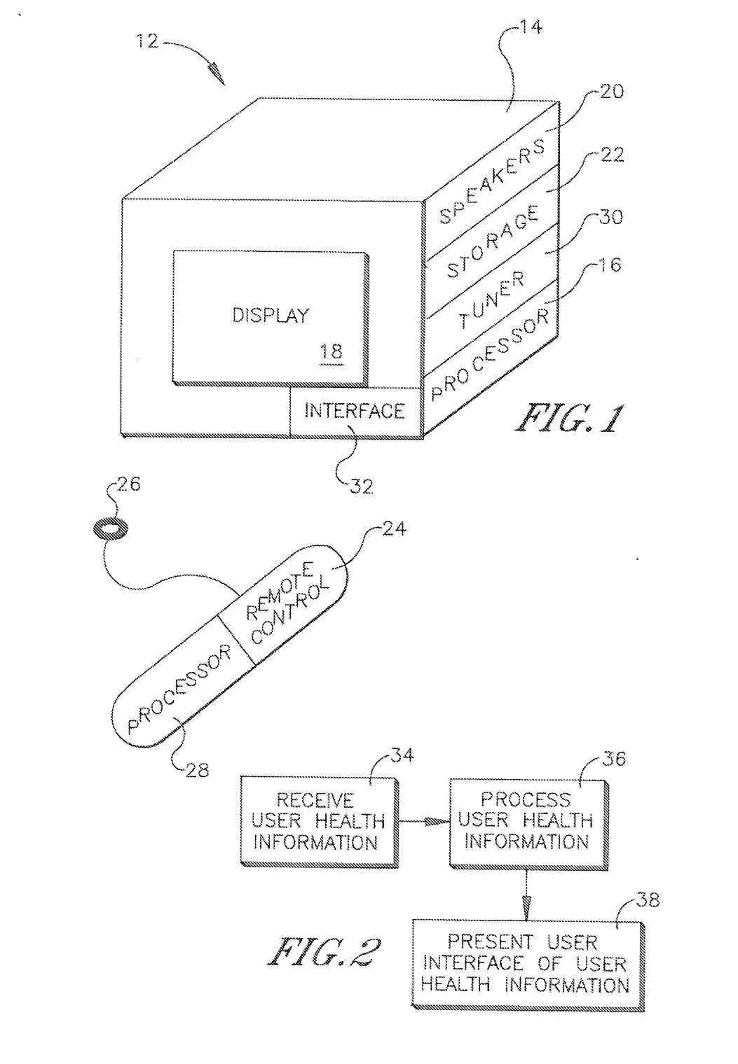

Referring initially to the non-limiting embodiment show in FIG. 1, a TV 12 includes a housing 14 bearing a digital processor 16. The processor 16 can control a visual display 18 and an audible display 20 such as one or more speakers. To undertake present principles, the processor 16 may access one or more computer readable data storages 22 such as but not limited to RAM-based storage (e.g., a chip implementing dynamic random access memory (DRAM)) or flash memory or disk-based-storage. Software code implementing present logic executable by the TV 12 may also be stored on one of the memories shown to undertake present principles.

The processor 16 can receive user health information from various input devices including a remote control device 24, a point and click device such as a mouse, a keypad, etc. The remote control device 24 may include a health monitoring sensor 26 and a processor 28. The sensor 26 may be, without limitation, a wrist band, strap or bracelet configured to be worn on a user's wrist. In non-limiting embodiments, the sensor 26 can communicate with the remote control device 24 over a wire (such as, e.g., a USB cable) or wirelessly (using, e.g., Bluetooth technology).

Further, the sensor 26 is to be understood to be capable of receiving and/or gathering user health information, which may include, but is not limited to, heart rate data, blood pressure data, blood glucose data, and body mass index (BMI) data. The sensor 26 is also understood to be capable of generating signals representing the gathered user health information which may then be sent to the remote control device 24.

In non-limiting embodiments such as the one shown in FIG. 1, both the TV 12 the remote control device 24 have IR codes representing health signals. Thus, the processor 28 of the remote control device 24 is capable of converting signals related to user health information generated by the sensor 26 into IR coded signals. The TV 12 can thus recognize IR coded signals of user health information received from the remote control 24.

Still in reference to FIG. 1, a TV tuner 30 may be provided to receive TV signals from a source such as a set-top box, satellite receiver, cable head end, terrestrial TV signal antenna, etc. Signals from the tuner 30 are sent to the processor 16 for presentation on the display 18 and speakers 20.

The TV 12 shown in FIG. 1 may be, in non-limiting embodiments, an internet protocol TV (IPTV) capable of accessing the Internet. Thus, as shown in FIG. 1, there may be a network interface 32 such as a wired or wireless modem or wireless telephony transceiver that may communicate with the processor 16 to provide connectivity to a wide area network such as the Internet.

Moving to FIG. 2, a flow chart of non-limiting exemplary logic for presenting user health information on a first UI is shown. Beginning at block 34, user health information sent from the remote control device 24 is received by the TV 12. At block 36 the logic may then process (e.g. store and arrange) user health information for presentation on a UI, the UI to be presented on the display 18. Concluding the non-limiting exemplary logic shown in FIG. 2, a visual representation of the user health information is presented on a UI at block 38. Further, it is to be understood that the non-limiting logic of FIG. 2 may be used for plural users on the TV 12.

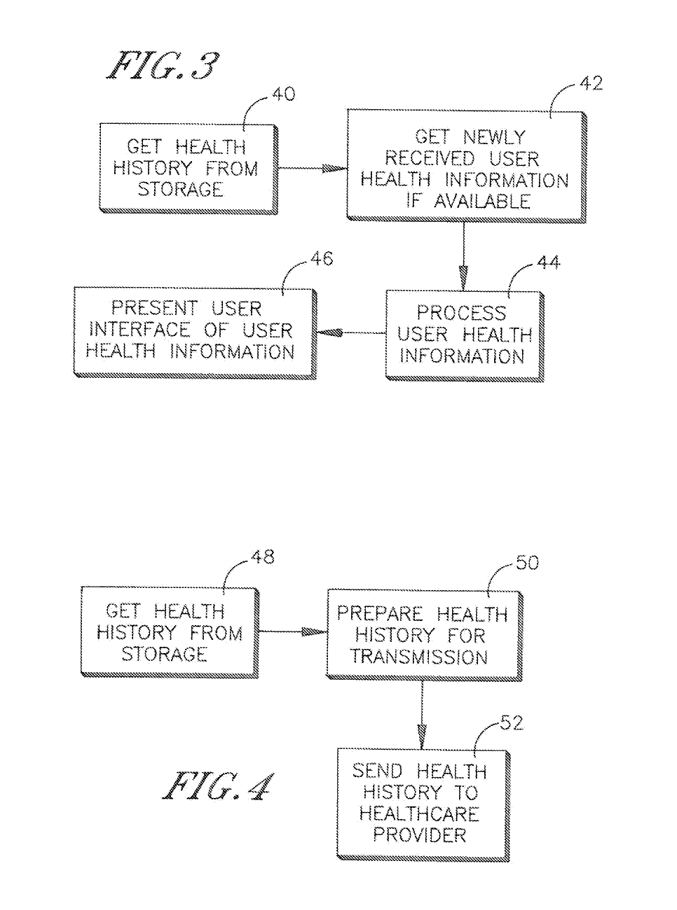

Now in reference to FIG. 3, a flow chart of the non-limiting exemplary logic for presenting user health information history on a UI is shown. Beginning at block 40, the logic may access user health information stored in the storage 22. The logic may then get newly received user health information at block 42 if such data is available. For example, newly received user health information may be received by the TV 12 while concurrently executing the logic process of FIG. 3. Thus, the newly received user health information may not have been stored with previously received user health information but may still be included in the user health information history to be presented on a UI, as will be described below.

Still in reference to FIG. 3, the logic then moves to block 44 where the logic may process (e.g. store and arrange) the user health information for presentation on a UI, the UI to be presented on the display 18. Concluding the non-limiting exemplary logic shown in FIG. 3, a visual representation of the user's health history associated with user health information is presented on a UI at block 46. Further, it is to be understood that the non-limiting logic of FIG. 3 may be used to maintain user health information history for plural users on the TV 12.

Moving on to FIG. 4, a flow chart of the non-limiting exemplary logic for sending user health information to a user's health care provider is shown. It is to be understood that the non-limiting exemplary logic shown in FIG. 4 may be instigated at the request of a user or may be performed automatically.

Beginning at block 48, the logic gets the user health information stored in the storage 22. Moving on to block 50, the logic may then prepare the user health information. In the non-limiting exemplary logic shown in FIG. 4, preparing user health information may include, but is not limited to, formatting user health information for transmission over the Internet, formatting user health information into a format acceptable for receipt by the health care provider, and arranging the user health information in chronological order. After the user health information has been prepared, the logic concludes at block 52 where the user health information is sent to a user's health care provider over the Internet. Alternatively, the information may be sent to a telephone number or to an email address (by, e.g., text entry) in other non-limiting embodiments. Further, it is to be understood that the non-limiting logic of FIG. 4 may be used to send user health information for plural users from the TV 12 to one or more health care providers.

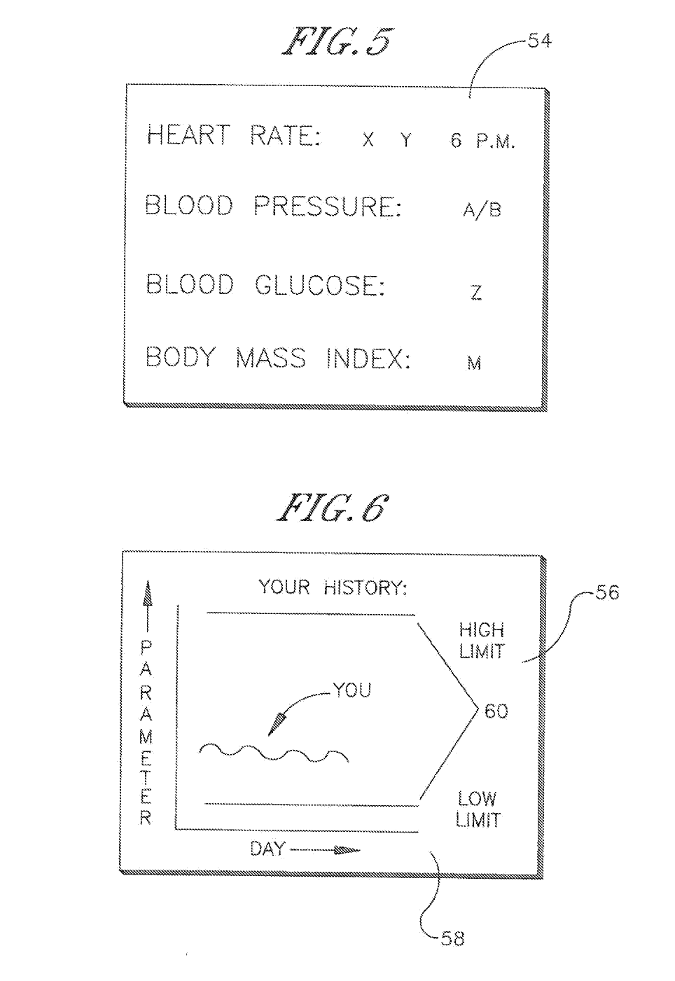

FIGS. 5 and 6 are non-limiting illustrative examples of screen shots of UIs that can be presented on the TV in accordance with present principles. FIG. 5 shows a UI 54 that may be presented on the display 18. The UI 54 may provide user health information relating to, e.g., heart rate, blood pressure, blood glucose, and BMI. It is to be understood that user health information on the UI 54 is exemplary and that more, less or different user health information may be displayed based upon user preference, available user health information, etc. Moreover, it is to be understood that the alphabetical variables shown in FIG. 5 are for illustrative purposes only and that numerical measurements are to be presented on the UI.

Moving to the UI of FIG. 6, a user may elect to invoke the UI 56 to view a history of various types of measurements such as heart rate history, blood pressure history, blood glucose history, and BMI history. As shown, in one embodiment the UI 56 presents a graph 58 of time (on the x-axis) versus health parameter value (on the y-axis) so that the user may see how particular health data varies over time. Also, upper and/or lower limit lines 60 may be provided which are established per standard of care guidance as to what the upper and/or lower “safe” limits of the parameter being displayed are. In this way, the user can compare his or her measured parameter against medically established guidelines.

While the particular USING TV AS HEALTH MONITOR is herein shown and described in detail, it is to be understood that the subject matter which is encompassed by the present invention is limited only by the claims.

1. A television (TV) comprising:
a housing;
a display on the housing;
a TV tuner;
a processor in the housing controlling the display and TV tuner;
an input device communicating with the processor, the input device having a health monitoring sensor attached thereto;
the processor executing logic including:
presenting a user interface (UI) on the display responsive to user health information gathered by the health monitoring sensor and received from the input device, wherein the user health information gathered by the health monitoring sensor includes blood glucose data.

2. The TV of claim 1, wherein the health monitoring sensor is a band configured to be worn on a person's wrist.

3. The TV of claim 1, wherein the health monitoring sensor is a strap-like bracelet configured to be worn on a person's wrist.

4. The TV of claim 1, wherein the input device has an processor which converts user health information gathered by the health monitoring sensor to infrared signals accepted by the TV.

5. The TV of claim 1, wherein user health information further includes heart rate data, blood pressure data, body mass index (BMI) data.

6. The TV of claim 1, wherein the UI is a first UI and the processor causes the first UI to present a visual representation of user health information.

7. The TV of claim 1, wherein the user health information received by the TV is stored in a storage area of the TV.

8. The TV of claim 7, wherein the processor causes a second UI to be presented on the display, wherein the second UI presents a visual representation of user health information history associated with stored user health information including heart rate data, blood pressure data, blood glucose data and/or BMI.

9. The TV of claim 8, wherein the second UI includes a graph of time on an x-axis versus parameter value on a y-axis.

10. The TV of claim 9, wherein the second UI presents upper and/or lower limit lines established per standard of care guidance as to what upper and/or lower “safe” limits of the health information being displayed are.

11. The TV of claim 1, wherein the TV is an internet protocol TV (IPTV) having a network interface so that the processor may communicate with the Internet through the network interface.

12. The TV of claim 11, wherein the TV sends current and/or stored user health information to a user's health care provider through the Internet.

13. A method, comprising:
receiving, at a TV, user health information from a health monitoring sensor via a TV remote control, the health information including a blood sugar level of the user;
storing the user health information; and
presenting on the TV a visualization of the user's health information history.

14. The method of claim 13, wherein the visualization is presented on a first UI.

15. The method of claim 13, wherein the health monitoring sensor gathers and/or receives user health information.

16. The method of claim 15, wherein the user health information further includes heart rate data, blood pressure data, BMI data.

17. The method of claim 13, wherein the health monitoring sensor is configured to be worn on a person's wrist.

18. The method of claim 13, wherein a second UI is presented on the display, the second UI providing a visual representation of user health information history associated with stored user health information.

19. The method of claim 13, wherein the TV is an internet protocol TV (IPTV) having a network interface for communication through the Internet.

20. The method of claim 19, comprising sending current and/or stored user health information to a user's health care provider through the Internet.

21. An apparatus comprising:
video display;
a TV tuner;
a processor controlling the display and TV tuner and communicating with the Internet through the network interface;
an input device communicating with the processor, the input device having a health monitoring sensor attached thereto, the health monitoring sensor being a blood glucose sensor;
the processor executing logic including:
receiving user health information from the health monitoring sensor;
processing the user health information; and
presenting on the display a visualization of the user health information.

22. The apparatus of claim 21, wherein the logic executed by the processor includes presenting on the display a history of user health information associated with current and stored user health information.

23. The apparatus of claim 21, wherein the user health information is sent to a user's health care provider through the Internet.
